(19) United States
(12) Patent Application Publication
(10) Pub. No.: US 2003/0097000 A1
(43) Pub. Date: May 22, 2003

(54) 2-Phenyl-quinoline derivatives, preparation method and therapeutic use thereof

(76) Inventors: Philippe R. Bovy, Mareil Marly (FR); Alain Braun, Boulogne Billancourt (FR); Christophe Philippo, Rueil-Malmaison (FR)

Correspondence Address: SANOFI-SYNTHELABO INC., 9 GREAT VALLEY PARKWAY, P.O. BOX 3026, MALVERN, PA, 19355, US

(21) Appl. No.: 10/149,840

(22) PCT Filed: Dec. 14, 2000
(86) PCT No.: PCT/FR00/03537

(30) Foreign Application Priority Data
Dec. 17, 1999 (FR) 99/15934

(51) Int. Cl. C 07D 4 1/02; C07D 215/20

(52) U.S. Cl. 546/153; 546/176

(57) ABSTRACT

Compounds of general formula (I)

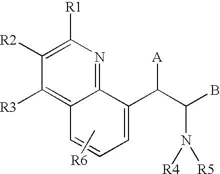

in which:

A represents a hydrogen atom or a hydroxyl,

B represents a hydrogen atom or a C₁₋₃ alkyl group,

R₁ represents a phenyl optionally substituted with a halogen, a hydroxyl, a C₁₋₃ alkoxy, C₁₋₃ alkyl, C₁₋₃ fluoroalkyl or C₁₋₂ perfluoroalkyl group,

R₂, R₃ and R₆, which may be identical or different, each represent a hydrogen atom, a halogen, a C₁₋₆ alkyl or C₂₋₆ alkenyl group,

R₄ and R₅, which may be identical or different, each represent a hydrogen atom, a C₁₋₆ alkyl, C₂₋₆ alkenyl or C₃₋₆ cycloalkyl group,

or R₄ and R₅ together form a C₂₋₆ alkylene or C₃₋₆ alkenylene chain to give, with the nitrogen to which they are attached, a heterocycle, this heterocycle optionally being substituted with a C₁₋₄ alkyl group.

Therapeutic use.

The present invention relates to 2-phenylquinoline derivatives, to preparations thereof and to therapeutic uses thereof.

The document “Chemical Abstracts Vol. 104, No. 13 of Mar. 31, 1986” cites the compounds RN69758-24-3, 6957-69-3 and 35871-03-5 of formula (I)

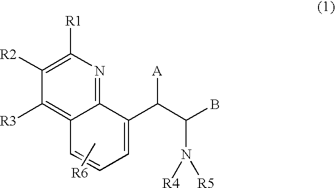

in which

A represents a hydroxyl;

B represents a hydrogen atom;

R₁ represents a 4-chlorophenyl;

R₂ represents a hydrogen atom;

R₃ represents a hydrogen;

R₄ and R₅ both represent an ethyl; and

R₆ represents a 6-chloro

or

A represents a hydroxyl;

B represents a hydrogen atom;

R₁ represents a phenyl;

R₂ and R₃ each represent a hydrogen atom;

R₄ and R₅ together form a C₆ alkylene chain; and

R₆ represents a hydrogen atom or a 6-methyl.

These compounds are active in the treatment of malaria.

The document from J-Gillepsie, J R et al. “Antimalarials. II. 8-quinolinemethanols” (JOURNAL OF MEDICINAL CHEMISTRY., Vol. 13, No. 5-1970, pages 860-864) discloses in table III thereof compounds 1b, 3a, 3b, 5b and of formula (I) in which:

A represents a hydroxyl;

B represents a hydrogen atom;

R₁ represents a 4-chlorophenyl;

R₂ represents a hydrogen atom; and

R₆ represents a 6-chloro, R₃ represents a hydrogen and R₄ and R₅ both represent an ethyl or a butyl; or

R₆ represents a 6-chloro, R₃ represents a methyl and R₄ and R₅ both represent a butyl; or

R₆ represents a hydrogen atom, R₃ represents a hydrogen and R₄ and R₅ both represent a butyl; or

R₆ represents a 5-chloro, R₃ represents a methyl and R₄ and R₅ both represent a butyl.

These compounds are active in the treatment of malaria.

The subjects of the present invention are 2-phenylquinoline derivatives corresponding to the general formula (I) below

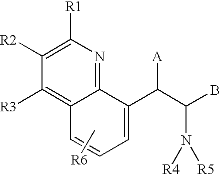

in which:

A represents a hydrogen atom or a hydroxyl,

B represents a hydrogen atom or a C₁₋₃ alkyl group,

R₁ represents a phenyl optionally substituted with a halogen, a hydroxyl, a C₁₋₃ alkoxy, C₁₋₃ alkyl, C₁₋₃ fluoroalkyl or C₁₋₂ perfluoroalkyl group,

R₂, R₃ and R₆, which may be identical or different, each represent a hydrogen atom, a halogen, a hydroxyl, a C₁₋₆ alkyl or C₂₋₆ alkenyl group,

R₄ and R₅, which may be identical or different, each represent a hydrogen atom, a C₁₋₆ alkyl, C₂₋₆ alkenyl or C₃₋₆ cycloalkyl group,

or R₄ and R₅ together form a C₂₋₆ alkylene or C₃₋₆ alkenylene chain to give, with the nitrogen to which they are attached, a heterocycle such as, for example, a piperidyl, azetidinyl or pyrrolidyl, this heterocycle optionally being substituted with one or two C₁₋₄ alkyl groups; and the salts or hydrates thereof,

with the exclusion of the compounds for which:

A represents a hydroxyl;

B represents a hydrogen atom;

R₁ represents a 4-chlorophenyl;

R₂ represents a hydrogen atom; and

R₆ represents a 6-chloro, R₃ represents a hydrogen and R₄ and R₅ both represent an ethyl or a butyl; or

R₆ represents a 6-chloro, R₃ represents a methyl and R₄ and R₅ both represent a butyl; or

R₆ represents a hydrogen atom, R₃ represents a hydrogen and R₄ and R₅ both represent a butyl; or

R₆ represents a 5-chloro, R₃ represents a methyl and R₄ and R₅ both represent a butyl;

and

A represents a hydroxyl;

B represents a hydrogen atom;

R₁ represents a phenyl;

R₂ and R₃ each represent a hydrogen atom;

R₄ and R₅ together form a C₂₋₆ alkylene chain; and

R₆ represents a hydrogen atom or a 6-methyl.

The preferred compounds according to the invention are those, as defined above, for which A represents a hydroxyl and more particularly the compounds for which A represents a hydroxyl and B represents a hydrogen atom.

Among these, the subgroups of compounds comprising radicals having the following meanings are preferred:

R₁ represents a phenyl optionally substituted with a halogen or a C₁₋₃ alkyl, C₁₋₃ alkoxy or C₁₋₂ perfluoroalkyl group, or

R₂ and R₃ represent, independently of each other, a hydrogen atom or a C₁₋₄ alkyl group, more preferably a methyl or ethyl, or

R₄ and R₅ each represent a C₁₋₄ alkyl group, more preferably a methyl, ethyl, propyl or isopropyl group, or

R₄ and R₅ together form a C₂₋₅ alkylene chain to give, with the nitrogen atom to which they are attached, a heterocycle, preferably an azetidinyl or a piperidyl, this heterocycle optionally being substituted with a C₁₋₂ alkyl group; or

R₆ represents a hydrogen atom.

More particularly, the preferred subgroup of compounds is that in which A represents a hydroxyl, B represents a hydrogen atom and R₁, R₂, R₃, R₄, R₅ and R₆ are as defined in the subgroups of preferred compounds.

In the context of the present invention, the compounds in the table are especially preferred, and more particularly the following:

2-Phenyl-3-methyl-8-(2-diethylamino-1-hydroxyethyl)-quinoline,

2-Phenyl-8-(2-ethylisopropylamino-1-hydroxyethyl)quinoline and

2-Phenyl-3-methyl-8-[2-(ethylisopropylamino)-1-hydroxyethyl]quinoline

Moreover, in the context of the present invention:

C_{1-z}(C_{2-z} or C_{2-z}), in which z can take values from 2 to 6, means a carbon chain that may contain from 1 (2 or 3) to z carbon atoms,

alkyl means a saturated linear or branched aliphatic group; for example a C₁₋₆ alkyl group represents a linear or branched carbon chain of 1 to 6 carbon atoms, more particularly a methyl, ethyl, propyl, isopropyl, butyl, isobutyl, tert-butyl, pentyl, etc.; preferably a methyl, ethyl, propyl or isopropyl,

fluoroalkyl means an alkyl in which one or two hydrogen atoms have been replaced with a fluorine atom,

perfluoroalkyl means an alkyl in which all the hydrogen atoms have been replaced with a fluorine atom,

cycloalkyl means a cyclic alkyl, for example a C₃₋₆ cycloalkyl group represents a cyclopropyl, cyclobutyl, cyclopentyl or a cyclohexyl,

alkenyl means a linear or branched monounsaturated or polyunsaturated aliphatic group preferably comprising 1 or 2 ethylenic unsaturations,

alkylene and alkenylene respectively mean a divalent alkyl and a divalent alkenyl,

alkoxy means an alkyloxy group containing a saturated linear or branched aliphatic chain, and

halogen atom means a fluorine, a chlorine, a bromine or an iodine.

The term protecting group Pg means a group that makes it possible firstly to protect a reactive function such as a hydroxyl or an amine during a synthesis, and secondly to regenerate the intact reactive function at the end of the synthesis. Examples of protecting groups and also protection and deprotection methods are given in Protective groups in Organic Synthesis, Greene et al., 2nd Ed. (John Wiley & Sons, Inc., New York). Moreover, in the case, for example, in which R₂, R₃ and R₆ represent such reactive functions, these radicals may be protected before reaction and deprotected according to these methods, and a person skilled in the art will readily determine the cases in which this protection is necessary.

The term leaving group means a group that can readily be cleaved from a molecule, with loss of an electron pair, by breaking a heterolytic bond. This group may thus be readily replaced with another group during a substitution reaction, for example. Such leaving groups are, for example, halogens or an activated hydroxyl group such as a mesyl, tosyl, triflate, acetyl, etc. Examples of leaving groups and also preparation references are given in “Advanced Organic Chemistry”, J. March, 3rd Edition, Wiley Interscience, pp. 310-316.

The compounds of general formula (I) may include one or more asymmetric carbon atoms. They may thus exist in the form of enantiomers or diastereoisomers. These enantiomers and diastereoisomers, and also mixtures thereof, including racemic mixtures, form part of the invention.

When a compound according to the invention shows stereoisomerism, for example of axial-equatorial or Z-E type, the invention comprises all the stereoisomers of these compounds.

The compounds of general formula (I) may be in the form of free bases or of addition salts with acids, which also form part of the invention. These salts, according to the present invention, comprise those with mineral or organic acids that allow a suitable separation or crystallization of the compounds of formula (I), such as picric acid, oxalic acid or an optically active acid, for example a tartaric acid, a dibenzoyltartaric acid, a mandelic acid or a camphorsulfonic acid, and those which form physiologically acceptable salts, such as the hydrochloride, hydrobromide, sulfate, hydrogen sulfate, dihydrogen phosphate, maleate, fumarate, pamoate, 2-naphthalenesulfonate or para-toluenesulfonate. Although the pharmaceutically acceptable salts are preferred, the other salts form part of the present invention. These salts may be prepared according to methods known to those skilled in the art, for example by reacting the compound of formula (I) in base form with the acid in a suitable solvent, such as an alcoholic solution or an organic solvent, followed by separation from the medium containing them by evaporating off the solvent or by filtration.

A second subject of the present invention is processes for preparing the 2-phenylquinoline derivatives of formula (I) according to the invention. They may be prepared according to various processes, especially those described below.

1. The compounds of formula (I), in particular those for which A represents a hydroxyl group, may be prepared according to reaction scheme 1.

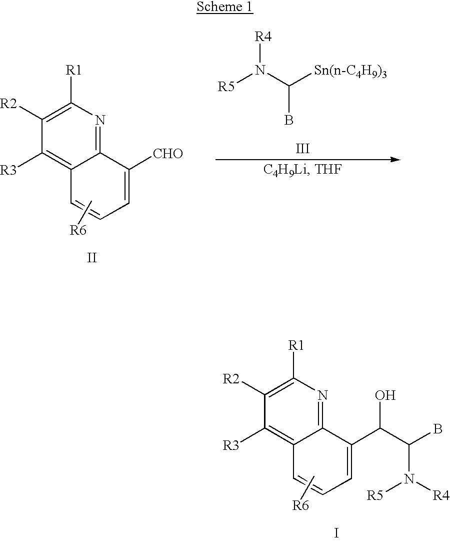

According to this process, an aldehyde of formula II is reacted with a stannate derivative of formula III. This reaction may be performed according to the method described by A. R. Katrizky et al. (Synthesis 1994; 907) in an organic ether solvent such as ether or tetrahydrofuran (THF), in the presence of n-butyllithium. The reaction is preferably performed at −78° C.

The meanings of R₁, R₂, R₃, R₄, R₅, R₆ and B in the compounds of formula II or III are those given in formula I.

The compounds of formula II may be prepared, according to scheme 2, by a formylation reaction of a quinoline derivative of formula IV, in which R₁, R₂, R₃ and R₆ are as defined in formula (I) and Y represents a leaving group such as, for example, a halogen, or an activated hydroxyl group such as a triflate group. The reaction may be performed by means of palladium catalysis according to the process described by Kotsuki H. et al. (Synthesis 1996, 470-472) or alternatively by lithiation of the quinoline derivative of formula IV and treatment with N,N-dimethylformamide (DMF).

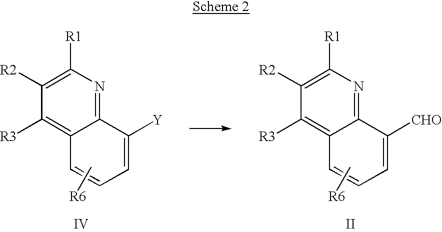

The compounds of formula III may be prepared by a person skilled in the art according to the process described by A. R. Katrizky et al. (Synthesis 1994; 907).

The compounds of formula IV may be synthesized according to methods known to those skilled in the art, especially those described in patent application PCT/FR99/02129. Other methods that have been used involve the processes described below.

Thus, the compounds of formula IV may be prepared by a Skraup or Doebner-Miller reaction, according to reaction scheme 3.

According to this scheme and under the conditions defined by Belser P. Tetrahedron 1996, Vol. 52, No 8, 2937-2944 or advantageously under the conditions defined by Z. Song, J. Heterocyclic Chem. 1993, 30, 17-21, an aniline of formula VII, for which Z represents a hydroxyl or methoxy group, and an α,β-unsaturated aldehyde or ketone of formula VI are heated in the presence of a dehydrating agent such as sulfuric acid and an oxidizing agent such as sodium iodide to form a quinoline derivative of formula V substituted in position 8 with a group Z. This compound is then treated with a phenyllithium derivative of formula VIII in a solvent such as toluene to give the compound of formula IX. The group Z of the compound thus obtained is then converted into a leaving group according to methods known to those skilled in the art. For example, when Z represents a methoxy group, this group is first converted into a hydroxyl group, for example in the presence of boron tribromide in a chlorinated solvent such as dichloromethane or chloroform, and then into a leaving group according to methods known to those skilled in the art to give the compound of formula IV in which Y represents a leaving group. The meanings of R₁, R₂, R₃ and R₆ in the compounds of formulae IV, V, VI, VII, VIII and IX are those given in formula I.

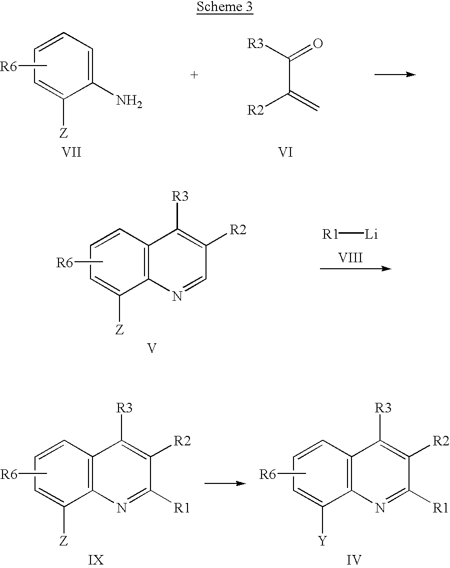

Moreover, the compounds of formula IV may be prepared by a Friedländer condensation reaction.

According to this process described in reaction scheme 4 and under the conditions defined by R. P. Thummel et al., J. Org. Chem. 1993, 58, 1666-1671, heating a phenyl ketone of formula XI, for which Y is as defined above, with a 2-acylaniline of formula X, in a high-boiling solvent such as toluene, and in the presence of alcoholic potassium hydroxide, gives the compounds of formula IV. The meanings of R₁, R₂, R₃ and R₆ in the compounds of formulae IV, X and XI are those given in formula I.

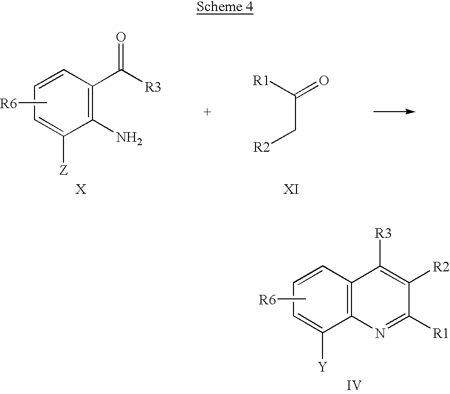

2. The compounds of formula (I) according to the invention, for which A is a hydroxyl group, may also be prepared according to reaction scheme 5.

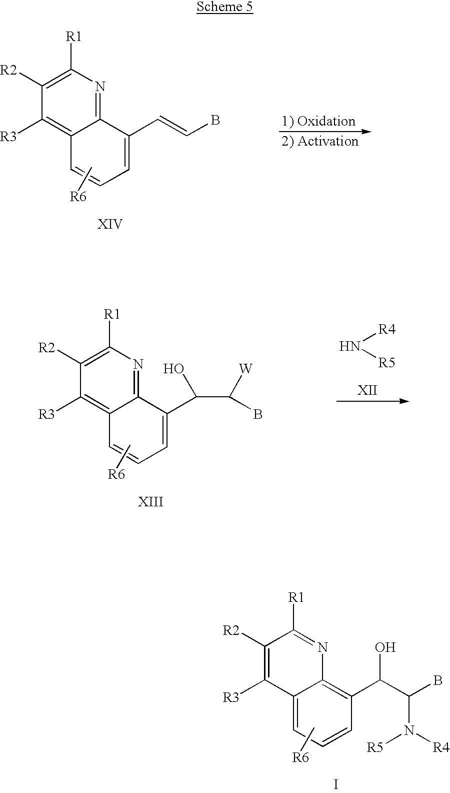

According to this scheme an ethenylquinoline derivative of formula XIV is reacted with an oxidizing agent such as sodium periodate, osmium tetroxide (in the racemic or chiral series by using AD-mix-α or AD-mix-β) or meta-chloroperbenzoic acid, followed by a hydrolysis in basic or acidic medium, so as to form a diol of formula XIII in which W represents a hydroxyl. The hydroxyl group geminal to the group B of the diol thus obtained may then be optionally selectively activated, in a manner that is known to those skilled in the art, so as to obtain the compound of formula XIII, in which W represents a leaving group, such as a tosyl group, an acetyl group or a bromine atom. The compound of formula (I) according to the invention is then prepared from the compound of formula XIII, by reacting it with an amine NHR₄R₅. The meanings of R₁, R₂, R₃, R₄, R₅, R₆ and B in each of the compounds of formula XIV or XIII and of the amine NHR₄R₅ are those given in formula (I).

The ethenylquinoline derivative of formula XIV may itself be prepared by a Stille palladium coupling, under the conditions defined by D. R. McKean et al. (J. Org. Chem., 52; 1987; 492) using a derivative of formula IV as defined above for which Y represents a leaving group, such as a halogen or an activated hydroxyl group, such as a triflate group.

Alternatively, the ethenylquinoline derivative of formula XIV may be prepared from an aldehyde derivative of formula II as defined above, by a Wittig reaction with the corresponding ylide (phenyl)₃P⁺—⁻CHB, under conditions that are standard for those skilled in the art.

3. The compounds of formula (I), in particular those for which A represents a hydroxyl group, may also be prepared, according to scheme 6, from a quinoline derivative of formula XV, for which Y represents a leaving group such as a halogen, for instance a bromine, an iodine or a chlorine, or an activated hydroxyl group such as a triflate group, by a palladium coupling, for example a Stille or Suzuki reaction, with a compound of formula R₁Sn(n—C₄H₉)₃ or R₁B(OH)₂, respectively, to give an intermediate compound of formula XVI, followed by opening of the epoxide of this compound according to methods known to those skilled in the art, for example in a solvent such as acetonitrile at temperatures of between 20 and 80° C. The meanings of B, R₁, R₂, R₃, R₄, R₅ and R₆ in the amine XII, the compound of formula XV and the compounds of formula R₁Sn(n—C₄H₉)₃ or R₁B(OH)₂ are those given in formula I.

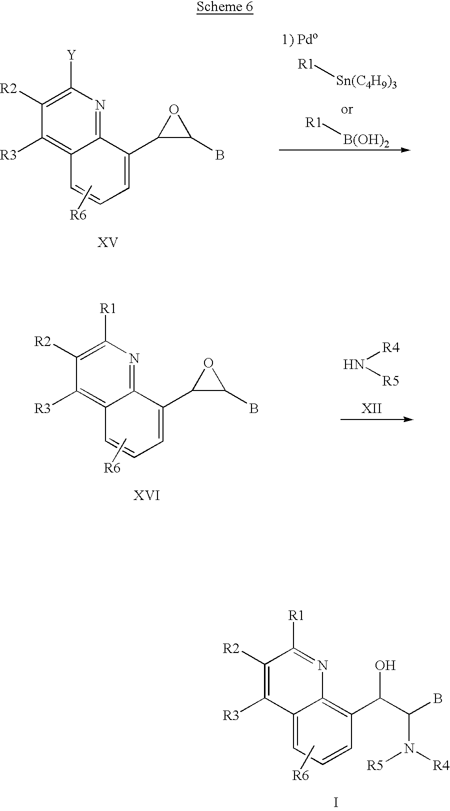

The compound of formula XV may itself be obtained according to reaction scheme 7.

According to this scheme, a quinoline of formula XVII is oxidized, by methods known to those skilled in the art, to an N-oxide compound of formula XVIII which, in the presence of acetic anhydride, and under the conditions defined in the patent by Tzeng, C. et al. U.S. Pat. No. 5,646,164, rearranges into a 2-acetoxyquinoline compound of formula XIX. The hydroxyl group in position 8 of this compound is converted into a leaving group such as a triflate group, and is then reacted with a vinylstannane compound of formula BCH═CH—Sn(n—C₄H₉)₃ under the conditions defined by McKean, D. R.; Parinello, G. Renaldo, A. F.; Stille, J. K., J. Org. Chem., 52, 1987, 492, to give the ethenyl derivative of formula XX, in which the acetoxy group in position 2 is then converted into a leaving group Y, such as a halogen, for instance a bromine, an iodine or a chlorine, or an activated hydroxyl group such as a triflate group, to give the ethenyl derivative of formula XXI. The quinoline derivative of formula XV can then be prepared by the action of a peracid such as meta-chloroperbenzoic acid on the quinoline of formula XXI. The reaction may be performed under the conditions known to those skilled in the art, for example in a chlorinated solvent such as dichloromethane or chloroform, preferably at temperatures from 20 to 80° C. The meanings of R₂, R₃, R₆ and B in the compounds of formulae XV, XVII, XVIII, XIX, XX and XXI are those given in formula I.

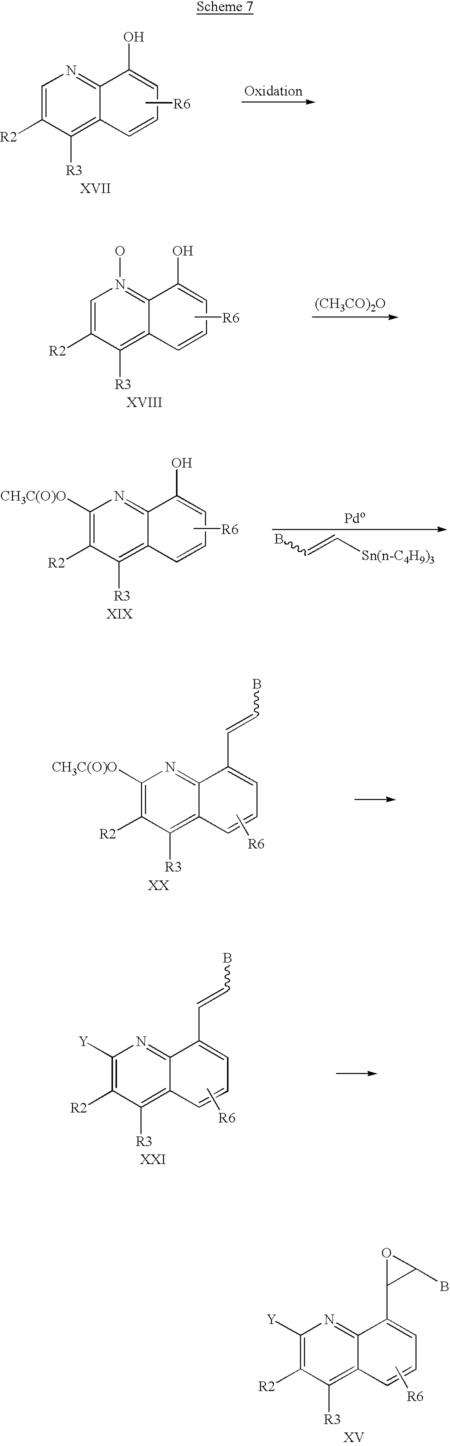

4. The compounds of formula (I) according to the invention, for which A represents a hydrogen atom, may be prepared by dehydroxylation of a corresponding compound of formula (I), in which A is a hydroxyl group.

The dehydroxylation reaction may be carried out, in a manner that is known to those skilled in the art, by reaction with triethylsilane and trifluoroacetic acid or according to the process described by A. G. Myers et al. (J. Am. Chem. Soc. 1997; 119; 8572-8573).

5. The compounds of formula (I) according to the invention, for which A is a hydrogen, may also be prepared according to the methods described in PCT/FR99/02129.

The starting compounds, especially the compounds of formulae VII, X, XI, XII and XVI are commercially available or may be prepared according to methods known to those skilled in the art.

The examples that follow are intended to illustrate the present invention without, however, limiting its scope.

### EXAMPLE 1

### 2-Phenyl-3-methyl-8-(2-diethylamino-1-hydroxyethyl)quinoline Hydrochloride

R₁=Phenyl; R₂=CH₃; R₃=H; R₄=R₅=C₂H₅; R₆=H; A=OH; B=H

(1) 3-Methoxy-2-aminobenzaldehyde

5 g of 3-methoxy-2-nitrobenzaldehyde are dissolved in a solution of 100 ml of ethanol, 100 ml of acetic acid and 50 ml of water. After addition of 1.4 g of iron and 1.4 ml of concentrated hydrochloric acid, the reaction medium is refluxed for 10-15 minutes. After cooling the reaction medium, 150 ml of water are added and the reaction mixture is extracted with 3×200 ml of dichloromethane. The organic phases are combined, washed with 500 ml of saturated sodium hydrogen carbonate solution, dried over magnesium sulfate, filtered and evaporated under vacuum to give 4.17 g of 3-methoxy-2-aminobenzaldehyde in the form of a colorless oil, which is used without purification for the following step. (Yield: quantitative)

(2) 2-Phenyl-3-methyl-8-methoxyquinoline

15.3 g (101 mol) of 3-methoxy-2-aminobenz-aldehyde and 14 ml (105 mol) of propiophenone are dissolved in 400 ml of ethanol. 1.4 g (25 mol) of potassium hydroxide are added to the reaction mixture, and this mixture is maintained at 100° C. for 8 hours. After cooling the reaction medium, it is concentrated under vacuum and 200 ml of water are added. A yellow precipitate forms after a few minutes. The medium is filtered and the yellow precipitate is taken up in a mixture of 300 ml of ethyl ether and 200 ml of 1N hydrochloric acid solution. The aqueous phase is extracted with 2×100 ml of ethyl ether. After addition of 300 ml of methylene chloride, 100 ml of 3N sodium hydroxide solution are then added to the aqueous phase. This phase is then extracted with 3×200 ml of methylene chloride. The organic phases are combined, dried over magnesium sulfate, filtered and concentrated under vacuum. 21.2 g of 2-phenyl-3-methyl-8-methoxyquinoline are obtained in the form of a pale yellow solid; m.p.=105° C. (Yield: 85%)

(3) 2-Phenyl-3-methyl-8-hydoxyquinoline

17.6 ml (180 mmol) of boron tribromide are added dropwise, at −30° C., to a solution of 21.2 g (85 mmol) of 2-phenyl-3-methyl-8-methoxyquinoline in 500 ml of methylene chloride. The cooling bath is then removed and the reaction medium is stirred for 3 hours until it has returned to room temperature. The reaction mixture is then poured onto ice and the medium is basified with sodium hydrogen carbonate. This mixture is then extracted with 3×200 ml of methylene chloride. The organic phases are combined, dried over magnesium sulfate, filtered and evaporated under vacuum. The residue is purified by chromatography on silica gel (eluent: 3/7 ethyl acetate/cyclohexane) to give 12.7 g of 2-phenyl-3-methyl-8-hydroxyquinoline in the form of a colorless oil. (Yield: 64%)

(4) 2-Phenyl-3-methyl-8-trifluoromethanesulfonato-quinoline

16.9 ml (110 mmol) of trifluoromethane sulfonic anhydride are added dropwise, at 0° C., to a solution of 12.5 g (53 mmol) of 2-phenyl-3-methyl-8-hydroxyquinoline in 150 ml of pyridine. The reaction mixture is then stirred for 16 hours at room temperature. After evaporating off the pyridine, the residue is taken up in 200 ml of water and 100 ml of ethyl acetate. The aqueous phase is extracted with 2×100 ml of ethyl acetate. The organic phases are combined, dried over magnesium sulfate, filtered and concentrated under vacuum. The residue is purified by chromatography on silica gel (eluent: 3/7 ethyl acetate/cyclohexane) to give 17.8 g of 2-phenyl-3-methyl-8-trifluoromethanesulfonatoquinoline in the form of a beige-colored solid; m.p.=85° C. (Yield: 91%)

(5) 2-Phenyl-3-methyl-8-vinylquinoline

6.3 g (150 mmol) of lithium chloride, 16.8 ml (57 mmol) of tributylvinyltin and 1.6 g (1.5 mmol) of Pd(PPh₃)₄ are sequentially added at room temperature to a solution of 17.7 g (48 mmol) of 2-phenyl-3-methyl-8-trifluoromethanesulfonatoquinoline in 250 ml of dioxane degassed beforehand with a flow of nitrogen. The reaction medium is then heated at 110° C. for 16 hours. After evaporating off the dioxane, the residue is taken up in 200 ml of water and 200 ml of ethyl acetate. The aqueous phase is extracted with ethyl acetate. The organic phases are combined, dried over magnesium sulfate, filtered and concentrated under vacuum. The residue is purified by chromatography on silica gel (eluent: 3/7 ethyl acetate/cyclohexane) to give 11.3 g of 2-phenyl-3-methyl-8-vinylquinoline in the form of a colorless oil. (Yield: 96%)

(6) 2-Phenyl-3-methyl-8-oxiranequinoline

0.5 g of 2-phenyl-3-methyl-8-vinylquinoline (20.3 mmol) and 10 ml of chloroform are placed in a 100 ml round-bottomed flask. The solution is cooled to 0° C. with an ice bath and 0.6 g (3.46 mmol) of meta-chloroperbenzoic acid are added. Stirring is continued for 2 hours at 0° C. and 15 ml of saturated sodium carbonate solution are added. The mixture is extracted with 3×50 ml of dichloromethane. The combined organic phases are dried over magnesium sulfate, filtered and concentrated under vacuum. The residue is purified by column chromatography on silica (elution solvent: 95/5 cyclohexane/ethyl acetate) to give 0.263 g of 2-phenyl-3-methyl-8-oxiranequinoline in the form of a colorless oil. (Yield: 50%)

(7) 2-Phenyl-3-methyl-8-(2-diethylamino-1-hydroxy-ethyl)quinoline

0.120 g of 2-phenyl-3-methyl-8-oxiranequinoline (0.46 mmol), 1 ml of diethylamine and 5 ml of chloroform are placed in a 100 ml three-necked flask equipped with a condenser. The solution is refluxed for 4 hours and then concentrated under vacuum. The residue is purified by column chromatography on silica (elution solvent: 95/5 dichloromethane/methanol) to give 0.124 g of 2-phenyl-3-methyl-8-(2-diethylamino-1-hydroxyethyl)quinoline in the form of a wax; 81% yield.

(8) 2-Phenyl-3-methyl-8-(2-diethylamino-1-hydroxy-ethyl)quinoline Hydrochloride

3.1 ml of hydrogen chloride as a 0.1M solution in isopropanol are added to 0.116 g (0.347 mmol) of 2-phenyl-3-methyl-8-(2-diethylamino-1-hydroxyethyl)quinoline obtained in step (7), and the salt is concentrated under vacuum. The residue is taken up in the minimum amount of ethyl ether and then filtered and dried in a desiccator under vacuum over phosphorus pentoxide. 0.1 g of 2-phenyl-3-methyl-8-(2-diethylamino-1-hydroxyethyl)quinoline hydrochloride is obtained; m.p. 208-210° C.

### EXAMPLE 2

### 2-Phenyl-8-[2-(ethylisopropylamino)-1-hydroxyethyl]quinoline Pamoate

R₁=Phenyl; R₂=R₃=H; R₄=Et, R₅=i-C₃H₇; R₆=H; A=OH; B=H

(1) 8-Hydroxyquinoline N-oxide

59.74 g (411 mmol) of 8-hydroxyquinoline, 350 ml (822 mmol) of dichloromethane, 82.2 ml of 35% aqueous hydrogen peroxide solution and 0.52 g (2.5 mmol) of methylrhenium trioxide (MTO) are placed in a 1 L round-bottomed flask. The reaction mixture is stirred at room temperature (25° C.) for 24 hours, followed by successive addition of 80 ml of aqueous hydrogen peroxide solution and 0.32 g of manganese dioxide. The mixture is stirred for 1 hour 30 minutes and the phases are then separated by settling. The aqueous phase is extracted with dichloromethane (2×200 ml). The organic phases are combined, dried over sodium sulfate, filtered and concentrated under vacuum to give 64 g of 8-hydroxyquinoline N-oxide in the form of an orange-colored solid; m.p.=112° C. (Yield: 97%)

(2) 2-Acetoxy-8-hydroxyquinoline

64 g (397 mmol) of 8-hydroxyquinoline N-oxide, 550 ml of acetic anhydride and 40 ml of acetic acid are placed in a 1 L round-bottomed flask. The reaction mixture is refluxed (135° C.) for 24 hours, a further 40 ml of acetic acid are then added and heating is continued for 1 hour 30 minutes. The reaction mixture is allowed to cool to room temperature and 400 ml of toluene are added. A precipitate appears and is filtered off. A further 400 ml of toluene are added and the mixture is filtered. The precipitate is washed with 200 ml of ethyl ether and is dried in a desiccator under vacuum over phosphorus pentoxide to give 70.5 g of 2-acetoxy-8-hydroxyquinoline in the form of a brown solid; m.p.=[lacuna] °C. (Yield: 88%)

(3) 2-Acetoxy-8-trifluoromethanesulfonatoquinoline

36 g (176 mmol) of 2-acetoxy-8-hydroxy-quinoline and 300 ml [lacuna] are placed in a 1 L round-bottomed flask. The reaction mixture is cooled with an ice bath and 62.3 ml (370 mmol) of trifluoromethane-sulfonic anhydride are added dropwise, and stirring is continued at 0° C. for 3 hours. The reaction mixture is poured onto a mixture of 200 ml of 3M hydrochloric acid and ice. A brown precipitate appears and is filtered off and then washed with 3×50 ml of water and is dried in a desiccator under vacuum over phosphorus pentoxide to give 56.6 g of 2-acetoxy-8-trifluoromethanesulfonatoquinoline in the form of a brown solid; m.p.=90° C. (Yield: 96%)

(4) 2-Acetoxy-8-vinylquinoline

33.6 g (100 mmol) of 2-acetoxy-8-trifluoromethanesulfonatoquinoline, 12.7 g of LiCl (300 mmol), 34.9 g (110 mmol) of tributylvinyltin and 5.8 g of tetrakis(triphenylphosphine)palladium are placed in a 1 L round-bottomed flask containing 300 ml of dioxane that has been degassed beforehand. The mixture is refluxed for 4 hours and then concentrated under vacuum and hydrolyzed by addition of 200 ml of water. The aqueous phase is extracted with ethyl acetate (4×200 ml). The organic phases are combined, dried over magnesium sulfate, filtered and concentrated under vacuum. The residue is purified by column chromatography on silica (eluent: 95/5 cyclohexane/ethyl acetate) to give a fraction containing 2-hydroxy-8-vinylquinoline and 18.6 g of 2-acetoxy-8-vinylquinoline in the form of a yellow oil. (Yield =87%)

(5) 2-Hydroxy-8-vinylquinoline

18.6 g (87.3 mmol) of 2-acetoxy-8-vinylquinoline, 290 ml of water and 290 ml of methanol are placed in a 1 L round-bottomed flask. The reaction mixture is heated at 55° C. for 2 hours and the methanol is then evaporated off under vacuum. The resulting mixture is extracted with ethyl acetate (2×200 ml). The organic phases are combined, dried over magnesium sulfate, filtered and concentrated under vacuum to give 15.0 g of 2-hydroxy-8-vinylquinoline in the form of a yellow solid; m.p.=96° C. (Quantitative yield)

(6) 2-Trifluoromethanesulfonato-8-vinylquinoline

7.5 g (43.86 mmol) of 2-hydroxy-8-vinylquinoline, 5.32 ml of pyridine (65.78 mmol) and 100 ml of dichloromethane are placed in a 250 ml round-bottomed flask. The mixture is cooled to 0° C. with an ice bath and 11.10 ml (65.78 mmol) of trifluoromethane-sulfonic anhydride are added dropwise. Stirring is continued at 0° C. for half an hour and the reaction mixture is hydrolyzed by addition of 100 ml of water. The resulting mixture is extracted with dichloromethane (3×150 ml). The organic phases are combined, dried over magnesium sulfate, filtered and concentrated under vacuum. The residue is purified by column chromatography on silica (eluent: 1/1 heptane/dichloromethane) to give 7.2 g of 2-trifluoromethane-sulfonato-8-vinylquinoline in the form of a yellow oil. (Yield=52%)

(7) 2-Trifluoromethanesulfonato-8-oxiranequinoline 1.85 g (6.1 mmol) of 2-trifluoromethane-sulfonato-8-vinylquinoline and 2.7 g (12.2 mmol) of meta-chloroperbenzoic acid are dissolved in 50 ml of chloroform in a 100 ml round-bottomed flask. The mixture is heated at 60° C. for 24 hours and then poured into saturated sodium bicarbonate solution. The resulting mixture is extracted with dichloromethane (3×150 ml). The organic phases are combined, dried over magnesium sulfate, filtered and concentrated under vacuum to give 1.75 g of 2-trifluoromethanesulfonato-8-oxiranequinoline in the form of an oil. (Yield=83%)

(9) 2-Phenyl-8-oxiranequinoline

243 mg (2.0 mmol) of phenylboronic acid, 207 mg (1.5 mmol) of potassium carbonate and 35 mg (0.3 mmol) of tetrakis(triphenylphosphine)palladium are introduced into a solution, degassed beforehand, of 0.319 g (1.0 mmol) of 2-trifluoromethanesulfonato-8-oxiranequinoline in 10 ml of toluene in a 20 ml three-necked flask. The mixture is heated on an oil bath at 60° C. for 4 hours and 30 ml of water are added. The resulting mixture is extracted with ethyl acetate and the organic phases are combined, dried over magnesium sulfate, filtered and concentrated under vacuum. The residue is purified by column chromatography on silica (eluent: 2/8 ethyl acetate/cyclohexane) to give 140 mg of 2-phenyl-8-oxiranequinoline in the form of a colorless oil. (Yield=55%)

(10) 2-Phenyl-8-[2-(ethylisopropylamino)-1-hydroxy-ethyl]quinoline

0.110 g of 2-phenyl-8-oxiranequinoline (0.44 mmol), 1.2 ml of ethylisopropylamine and 10 ml of chloroform are placed in a 100 ml three-necked flask equipped with a condenser. The solution is heated at 60° C. for 4 hours and then concentrated under vacuum. The residue is purified by column chromatography on silica (elution solvent: 95/5 dichloromethane/methanol) to give 0.140 g of 2-phenyl-8-[2-(ethylisopropylamino)-1-hydroxyethyl]quinoline in the form of a wax. (Yield 95%)

(11) 2-Phenyl-8-[2-(ethylisopropylamino)-1-hydroxy-ethyl]guinoline pamoate

A solution of 129 mg (0.33 mmol) of pamoic acid in 2 ml of DMF is added to a solution of 120 mg (0.33 mmol) of 2-phenyl-8-(1-diethylamino-2-hydroxyethyl)quinoline in 2 ml of DMF. The solution is stirred for 15 minutes and then 15 ml of distilled water are added. The yellow precipitate obtained is filtered off, washed with 5×10 ml of distilled water and then dried in a desiccator under vacuum over phosphorus pentoxide to give 248 mg of 2-phenyl-8-[2-(ethylisopropylamino)-1-hydroxyethyl]quinoline pamoate in the form of a yellow solid; m.p.=110° C.

Quantitative Yield

The table below illustrates the chemical structures and the chemical properties of some of the compounds of formula (I) according to the invention. These compounds were synthesized according to the methods described above.

TABLE

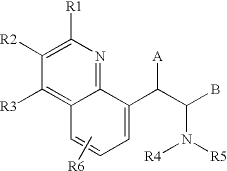

| | | | | | | | | | | m.p. |
|---|---|---|---|---|---|---|---|---|---|---|
| No. | R₁ | R₂ | R₃ | R₄ | R₅ | R₆ | A | B | Salt | (° C.) |
| 1 | Ph | Me | H | Et | Et | H | OH | H | — | oil |
| 2 | Ph | Me | H | Et | Et | H | OH | H | HCl | 208- |
| | | | | | | | | | | 210 |
| 3 | Ph | Me | H | —CH(Me)(CH₂)₃ | | H | OH | H | — | oil |
| | | | | CH(Me)- | | | | | | |
| 4 | Ph | Me | H | —CH(Me)(CH₂)₃ | | H | OH | H | HCl | >250 |
| | | | | CH(Me)- | | | | | | |
| 5 | Ph | Me | H | i-Pr | Et | H | OH | H | — | oil |
| 6 | Ph | Me | H | i-Pr | Et | H | OH | H | pam. | 216- |
| | | | | | | | | | | 218 |
| 7 | Ph | Me | H | —CH(Me)(CH₂)— | | H | OH | H | — | oil |
| 8 | Ph | Me | H | —CH(Me)(CH₂)— | | H | OH | H | pam. | 208- |
| | | | | | | | | | | 218 |
| 9 | Ph | H | H | Et | Et | H | OH | H | — | oil |
| 10 | Ph | H | H | Et | Et | H | OH | H | pam. | 120 |
| 11 | Ph | H | H | Et | i-Pr | H | OH | H | — | oil |
| 12 | Ph | H | H | Et | i-Pr | H | OH | H | pam. | 110 |
| 13 | Ph(2-CF₃) | H | H | Et | Et | H | OH | H | — | oil |
| 14 | Ph(2-CF₃) | H | H | Et | Et | H | OH | H | pam. | 120 |
| 15 | Ph(4-CF₃) | H | H | Et | Et | H | OH | H | — | oil |
| 16 | Ph(4-CF₃) | H | H | Et | Et | H | OH | H | pam. | 115 |
| 17 | Ph(2-OMe) | H | H | Et | Et | H | OH | H | — | oil |
| 18 | Ph(2-OMe) | H | H | Et | Et | H | OH | H | pam. | 108- |
| | | | | | | | | | | 113 |
| 19 | Ph(3-OMe) | H | H | Et | Et | H | OH | H | — | oil |
| 20 | Ph(3-OMe) | H | H | Et | Et | H | OH | H | pam. | 112- |
| | | | | | | | | | | 114 |
| 21 | Ph(2-Cl) | H | H | Et | Et | H | OH | H | — | oil |
| 22 | Ph(2-Cl) | H | H | Et | Et | H | OH | H | pam. | |
| 23 | Ph(2-Cl) | Me | H | Et | Et | H | OH | H | — | oil |
| 24 | Ph(2-Cl) | Me | H | Et | Et | H | OH | H | pam. | |
| 25 | Ph(2-Me) | Me | H | Et | Et | H | OH | H | — | oil |
| 26 | Ph(2-Me) | Me | H | Et | Et | H | OH | H | pam. | 124- |
| | | | | | | | | | | 134 |

In this table:
pam. represents a pamoic acid salt,
HCl represents a hydrochloride,
“—” represents a compound in free form,
Ph represent a phenyl,
i-Pr represents an isopropyl group,
Et represents an ethyl group,
Me represents a methyl group.

The compounds of the invention were subjected to biological tests intended to demonstrate their selective contractile activity on smooth muscle.

1. The in vitro activity of the compounds of the invention was studied on urethral and arterial smooth muscles.

These tests were carried out on female New Zealand rabbits weighing from 3 to 3.5 kg. The animals were killed by vertebral dislocation, and rings of urethral and mesenteric artery tissue were then taken. These rings of tissue were immersed in a modified Krebs solution, oxygenated with a mixture of 95% O₂ and 5% CO₂. Each sample of tissue was subjected to a tension of 1 g, after which phenylephrine was introduced at cumulative doses and the dose/response curve was established. After rinsing the samples, the compound to be studied was introduced at cumulative doses and the dose/response curve was established. The contractile effect of each compound is evaluated by calculating the pD₂ (negative logarithm of the agonist concentration which induces 50% of the maximum contraction) as well as by the maximum effect representing the percentage of the maximum contraction obtained with phenylephrine (% Eₘₐₓ).

The results obtained show that the compounds in accordance with the invention have:

a urethral pD₂ of greater than 2.5, usually between 4 and 8 and more generally between 5 and 8,

an arterial pD₂ of less than 3,

a urethral %Eₘₐₓ of greater than 30, usually between 40 and 90,

an arterial %Eₘₐₓ equal to zero.

2. The in vivo activity of the compounds of the invention on urethral and blood pressure was studied in demyelinized rats and in rabbits, according to the following protocols:

Pithed Rats

Wistar rats are anesthetized and pithed (according to the technique described by Gillespie, MacLaren A. and Polock D., A method of stimulating different segments of the autonomic outflow from the spinal column to various organs in the pithed cat and rat; Br. J. Pharmacol., 1970, 40: 257-267).

Catheters are introduced via the aorta and a jugular vein. Another catheter is introduced into the urethra via an incision made in the bladder. The test compounds are administered at increasing doses by intravenous perfusion.

The results are expressed as doses (μg/kg) required to increase the urethral pressure by 10 cm of water (UP₁₀) or the arterial pressure by 10 mmHg (AP₁₀) or by 50 mmHg (AP₅₀).

The compounds of the invention thus tested made it possible to obtain:

a UP₁₀ with doses of less than 100 μg/kg, usually between 20 and 100 μg/kg,

an AP₁₀ with doses of greater than 110 μg/kg, usually between 130 and 250 μg/kg,

the AP₅₀ could not be reached.

Rabbits

The experiments were performed on female New Zealand rabbits weighing between 3 and 4 kg, anesthetized with pentobarbital. Catheters are introduced for the descending aorta into the femoral artery, into a jugular vein and into the urethra (1.5 cm under the neck of the bladder).

The test compounds are administered 5 to 15 days after the operation, by intravenous (i.v.) administration.

The compounds are administered via the i.v. route over 5 minutes, in a single dose (100 μg/kg).

The increase in urethral pressure (UP) and in arterial pressure (AP) was measured herein, relative to the basal urethral or arterial pressure, respectively.

The results obtained are expressed as a percentage of premedication values at 5 minutes after assay.

The compounds of the invention thus tested allowed an increase in the UP of greater than 70%, usually between 90 and 125%. The increase in the AP was always less than 10% and was usually 0%.

The above set of results shows that the compounds of the invention have strong urethral action and weak arterial action.

They may be used as medicinal products, in particular as agents for contracting smooth muscle, and even more particularly in the treatment of urinary incontinence, especially urinary exertion incontinence. In this indication, the compounds according to the invention show good efficacy and, usually, lower side effects than the medicinal products conventionally used for such a treatment, especially as regards the side effects affecting the arteries.

The compounds of the invention were subjected to biological tests intended to demonstrate their vasoconstrictive activity.

The in vitro activity of the compounds of the invention was studied on the saphene veins of Yucatan miniature pig. The tissue is cut into a spiral and is mounted in an isolated organ tank in a modified Krebs solution oxygenated with a mixture of 95% O₂ and 5% CO₂ maintained at 37° C. The blood vessel is linked to an isometric sensor under a basal tension of 1 g and is connected to a polygraph for recording the variations in tension. The viability of each preparation is tested by pre-stimulation with 3 μM noradrenalin. After rinsing, the test compound is introduced and its concentration-response curve is constructed cumulatively until a maximum response is obtained. The contractile effect of each compound is evaluated by calculating the EC₅₀ of the (concentration producing 50% of the maximum response).

The compounds of the invention made it possible to obtain vasoconstrictive activity with an EC₅₀ value usually of between 1 μM and 100 μM.

The compounds of the invention may be used in the treatment of venous insufficiency and venous ulcers.

The compounds according to the invention may also be used for the treatment of migraine, gastrointestinal disorders and as vasoconstrictors of nasal mucosa.

The use of the compounds according to the invention for the preparation of a medicinal product intended for treating the pathologies mentioned above forms an integral part of the invention.

According to another of its aspects, the present invention relates to pharmaceutical compositions containing a compound according to the invention as active principle.

Thus, these pharmaceutical compositions contain an effective dose of a compound according to the invention or of a pharmaceutically acceptable salt or hydrate thereof, and one or more pharmaceutically acceptable excipients.

Said excipients are chosen according to the desired pharmaceutical form and the desired mode of administration.

In the pharmaceutical compositions of the present invention for oral, sublingual, subcutaneous, intramuscular, intravenous, topical, intratracheal, intranasal, transdermal or rectal administration, the active principle of formula (I) above or its possible salt or hydrate can be administered in unit administration form, mixed with conventional pharmaceutical excipients, to animals and human beings for the prophylaxis or treatment of the above disorders or diseases. The appropriate unit administration forms comprise oral-route forms such as tablets, gel capsules, powders, granules and oral solutions or suspensions, sublingual, buccal, intratracheal and intranasal administration forms, subcutaneous, intramuscular or intravenous administration forms and rectal administration forms. The compounds according to the invention can be used in creams, ointments or lotions for topical application.

In order to obtain the desired prophylactic or therapeutic effect, the dose of active principle can vary between 0.1 μg and 50 mg per kg of body weight and per day. Although these dosages are examples of an average situation, there may be particular cases in which higher or lower dosages are appropriate, and such dosages also form part of the invention. According to the usual practice, the dosage which is appropriate for each patient is determined by the doctor according to the mode of administration and the weight and response of said patient.

Each unit dose can contain from 0.1 to 1000 mg, preferably from 1 to 500 mg, of active principle combined with a pharmaceutical excipient. This unit dose can be administered 1 to 5 times a day so as to administer a daily dosage of from 0.5 to 5000 mg, preferably from 1 to 2500 mg.

For example, when a solid composition in the form of tablets is prepared, the main active ingredient is mixed with a pharmaceutical excipient, such as gelatin, starch, lactose, magnesium stearate, talc, gum arabic or the like. The tablets can be coated with sucrose, a cellulose derivative or other materials. The tablets can be made via different techniques: direct tableting, dry granulation, wet granulation or hot melting.

According to a second example, a preparation as gel capsules is obtained by mixing the active ingredient with a diluent and pouring the mixture obtained into soft or hard gel capsules.

Aqueous suspensions, isotonic saline solutions or sterile, injectable solutions which contain pharmacologically compatible dispersants and/or wetting agents, for example propylene glycol or butylene glycol, are used for parenteral administration.

According to another of its aspects, the present invention also relates to a method for treating the pathologies indicated above, which comprises the administration of a compound according to the invention or a salt or hydrate thereof.

1. A compound of formula (I) 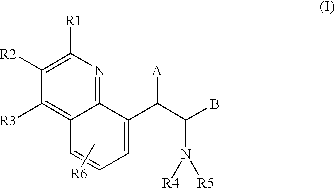
in which:
A represents a hydrogen atom or a hydroxyl,
B represents a hydrogen atom or a C₁₋₃ alkyl group,
R₁ represents a phenyl optionally substituted with a halogen, a hydroxyl, a C₁₋₃ alkoxy, C₁₋₃ alkyl, C₁₋₃ fluoroalkyl or C₁₋₂ perfluoroalkyl group,
R₂, R₃ and R₆, which may be identical or different, each represent a hydrogen atom, a halogen, a hydroxyl, a C₁₋₆ alkyl or C₂₋₆ alkenyl group,
R₄ and R₅, which may be identical or different, each represent a hydrogen atom, a C₁₋₆ alkyl, C₂₋₆ alkenyl or C₃₋₆ cycloalkyl group,
or R₄ and R₅ together form a C₂₋₆ alkylene or C₃₋₆ alkenylene chain to give, with the nitrogen to which they are attached, a heterocycle, this heterocycle optionally being substituted with one or two C₁₋₄ alkyl groups; and the salts or hydrates thereof,
with the exclusion of the compounds for which:
A represents a hydroxyl;
B represents a hydrogen atom;
R₁ represents a 4-chlorophenyl;
R₂ represents a hydrogen atom; and
R₆ represents a 6-chloro, R₃ represents a hydrogen and R₄ and R₅ both represent an ethyl or a butyl; or
R₆ represents a 6-chloro, R₃ represents a methyl and R₄ and R₅ both represent a butyl; or
R₆ represents a hydrogen atom, R₃ represents a hydrogen and R₄ and R₅ both represent a butyl; or
R₆ represents a 5-chloro, R₃ represents a methyl and R₄ and R₅ both represent a butyl; and
A represents a hydroxyl;
B represents a hydrogen atom;
R₁ represents a phenyl;
R₂ and R₃ each represent a hydrogen atom;
R₄ and R₅ together form a C₂₋₆ alkylene chain; and
R₆ represents a hydrogen atom or a 6-methyl.

2. The compound of formula (I) as claimed in claim 1, characterized in that
A represents a hydroxyl;
B represents a hydrogen atom;
R₁ represents a phenyl optionally substituted with a halogen or a C₁₋₃ alkyl, C₁₋₃ alkoxy or C₁₋₂ perfluoroalkyl group,
R₂ and R₃ represent, independently of each other, a hydrogen atom or a C₁₋₄ alkyl group,
R₄ and R₅ each represent a C₁₋₄ alkyl group, or R₄ and R₅ together form a C₂₋₄ alkylene chain to give, with the nitrogen atom to which they are attached, an azetidinyl or a piperidyl, this azetidinyl or piperidyl optionally being substituted with a C₁₋₂ alkyl group; and
R₆ represents a hydrogen atom.

3. The compound of formula (I) as claimed in either of claims 1 and 2, characterized in that it consists of:
2-Phenyl-3-methyl-8-(2-diethylamino-1-hydroxyethyl)-quinoline or
2-Phenyl-8-[2-(ethylisopropylamino)-1-hydroxyethyl]quinoline or
2-Phenyl-3-methyl-8-[2-(ethylisopropylamino)-1-hydroxyethyl]quinoline.

4. A process for preparing a compound of formula (I) as claimed in either of claims 1 and 2, characterized in that an aldehyde of formula II 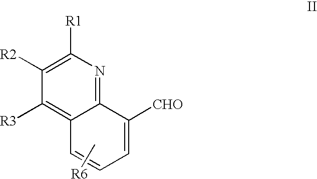
is reacted with a stannate derivative of formula III 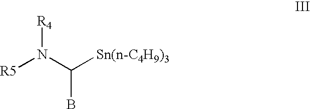
the meanings of R₁, R₂, R₃, R₄, R₅, R₆ and B of the aldehyde of formula II and of the stannate derivative of formula III being those defined for the compound of formula (I) as claimed in claim 1, to give the compound of formula (I) in which A represents a hydroxyl group, and the product is optionally dehydroxylated to give the compound of formula (I) in which A represents a hydrogen atom.

5. A process for preparing a compound of formula (I) as claimed in claim 1, in which A is a hydroxyl group, characterized in that an oxirane derivative of formula XVI 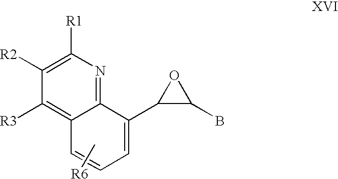
is reacted with an amine NHR₄R₅, the meanings of R₁, R₂, R₃, R₆ and B of the oxirane of formula XVI and of R₄ and R₅ of said amine being those defined for the compound of formula (I) as claimed in claim 1.

6. A process for preparing a compound of formula (I) as claimed in claim 1, in which A represents a hydroxyl, characterized in that a compound of formula XIII 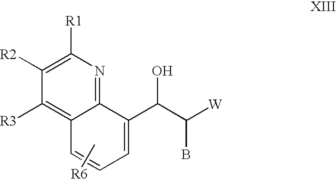
in which W represents an activated hydroxyl group, is reacted with an amine NHR₄R₅, the meanings of R₁, R₂, R₃, R₄, R₅, R₆ and B of the compound of formula XIII and of said amine being those defined for the compound of formula (I) as claimed in claim 1.

7. A pharmaceutical composition,
characterized in that it comprises a compound as claimed in one of claims 1, 2 and 3 and one or more suitable excipients.

8. The use of a compound as claimed in one of claims 1, 2 and 3, or of a compound of formula (I) as claimed in claim 1, in which
A represents a hydroxyl;
B represents a hydrogen atom;
R₁ represents a 4-chlorophenyl;
R₂ represents a hydrogen atom; and
R₆ represents a 6-chloro, R₃ represents a hydrogen and R₄ and R₅ both represent an ethyl or a butyl; or
R₆ represents a 6-chloro, R₃ represents a methyl and R₄ and R₅ both represent a butyl; or
R₆ represents a hydrogen atom, R₃ represents a hydrogen and R₄ and R₅ both represent a butyl; or
R₆ represents a 5-chloro, R₃ represents a methyl and R₄ and R₅ both represent a butyl; and
A represents a hydroxyl;
B represents a hydrogen atom;
R₁ represents a phenyl;
R₂ and R₃ each represent a hydrogen atom;
R₄ and R₅ together form a C₂₋₆ alkylene chain; and
R₆ represents a hydrogen atom or a 6-methyl;
for the preparation of a medicinal product intended for treating urinary incontinence, venous insufficiency, migraine or gastrointestinal disorders.
